(19) United States
(12) Patent Application Publication
(10) Pub. No.: US 2025/0115122 A1
(43) Pub. Date: Apr. 10, 2025

(54) VEHICLE OCCUPANT INTOXICATION DETECTION SYSTEMS AND METHODS

(71) Applicant: TOYOTA MOTOR ENGINEERING & MANUFACTURING NORTH AMERICA, INC., Plano, TX (US)

(72) Inventors: Brian A. Mulrooney, Brighton, MI (US); Daniel James Walter, Ypsilani, MI (US); Gustavo Muller Nunes, South Lyon, MI (US); Joshua D. Yaksich, Jackson, MI (US); Stuart Wuerthele, Saline, MI (US); William Fanelli, Ypsilani, MI (US); Naohiro Matsumura, Dallas, TX (US); Sergei Gage, Dallas, TX (US); Pujitha Gunaratne, Northville, MI (US); Jennifer Cohen Lerman, Dallas, TX (US); Mike Kushnerik, The Colony, TX (US); Schuyler St. Lawrence, Annandale, VA (US); Swathi Manoravi, Tamil Nadu (IN); Aarthi Ranganathan, Tamil Nadu (IN); Dharm Sidhu, Plano, TX (US)

(73) Assignees: TOYOTA MOTOR ENGINEERING & MANUFACTURING NORTH AMERICA, INC., Plano, TX (US); TOYOTA JIDOSHA KABUSHIKI KAISHA, Aichi-ken (JP)

(21) Appl. No.: 18/483,239

(22) Filed: Oct. 9, 2023

(51) Int. Cl. B60K 28/06; B60W 50/08; G01N 33/497; G01N 33/98

(52) U.S. Cl. CPC B60K 28/063; B60W 50/08; G01N 33/4972; G01N 33/98

(57) ABSTRACT

Systems and methods related to intoxication detection of one or more vehicle occupants are provided. A system includes a plurality of sensor assemblies disposed at different locations within a vehicle. The system further includes a controller configured to determine which occupant among a plurality of occupants in the vehicle is intoxicated based on an analysis of sensor signals received from the plurality of sensor assemblies. The controller is further configured to output an alert based on determining that a driver of the vehicle is intoxicated. Associated vehicles, controllers, and methods are also provided.

### TECHNICAL FIELD

The present disclosure relates generally to driver safety, and, more particularly, to systems and methods related to vehicle occupant intoxication detection.

### BACKGROUND

Conventional systems may identify intoxicated drivers using image sensors. However, these systems may not adequately identify intoxicated drivers or require expensive hardware to be installed in the vehicle. Therefore, a need exists for improved vehicle occupant intoxication detection.

### BRIEF SUMMARY

Various embodiments of the present disclosure include a system. The system includes a plurality of sensor assemblies disposed at different locations within a vehicle. The system further includes a controller configured to determine which occupant among a plurality of occupants in the vehicle is intoxicated based on an analysis of sensor signals received from the plurality of sensor assemblies. The controller is further configured to output an alert based on determining that a driver of the vehicle is intoxicated.

Various embodiments of the present disclosure include a vehicle. The vehicle includes a cabin, a plurality of sensor assemblies disposed at different locations within a cabin, and a controller. The controller is configured to determine which occupant among a plurality of occupants in the cabin is intoxicated based on an analysis of sensor signals received from the plurality of sensor assemblies. The controller is further configured to output an alert based on determining that a driver of the vehicle is intoxicated.

Various embodiments of the present disclosure include a controller. The controller is configured to determine which occupant among a plurality of occupants in a vehicle is intoxicated based on an analysis of sensor signals received from a plurality of sensor assemblies disposed at different locations within the vehicle. The controller is further configured to output an alert based on determining that a driver of the vehicle is intoxicated.

The scope of the invention is defined by the claims, which are incorporated into this section by reference. A more complete understanding of embodiments of the present invention will be afforded to those skilled in the art, as well as a realization of additional advantages thereof, by a consideration of the following detailed description of one or more embodiments. Reference will be made to the appended sheets of drawings that will first be described briefly.

### BRIEF DESCRIPTION OF THE DRAWINGS

FIG. 1 is a diagram illustrating various systems of a vehicle, according to one or more embodiments of the disclosure.

FIG. 2 is a diagram illustrating a sensor assembly placement within a vehicle, according to one or more embodiments of the disclosure.

FIG. 3 is a diagram illustrating vehicle restriction levels for a vehicle, according to one or more embodiments of the disclosure.

FIG. 4 is a diagram illustrating a sensor assembly, according to one or more embodiments of the disclosure.

FIG. 5 is a diagram illustrating a transparent view of the sensor assembly of FIG. 4, according to one or more embodiments of the disclosure.

FIG. 6 is a diagram illustrating front and rear perspective views of a sensor module of a sensor assembly, according to one or more embodiments of the disclosure.

FIG. 7 is a flowchart of a method of detecting an intoxication level of one or more vehicle occupants, according to one or more embodiments of the disclosure.

FIG. 8 is a diagram illustrating an example computing or processing system, according to one or more embodiments of the disclosure.

FIG. 9 is a diagram illustrating an example system, according to one or more embodiments of the disclosure.

FIG. 10 is a diagram illustrating example sensor readings of an intoxication sensor assembly, according to one or more embodiments of the disclosure.

Embodiments of the present disclosure and their advantages are best understood by referring to the detailed description that follows. It is noted that sizes of various components and distances between these components are not drawn to scale in the figures. It should be appreciated that like reference numerals are used to identify like elements illustrated in one or more of the figures.

### DETAILED DESCRIPTION

Embodiments of the present disclosure are directed to systems and methods related to identifying intoxicated drivers using in-cabin sensors and adjusting vehicle performance and/or parameters based on a detected intoxication. When the driver is determined as intoxicated, an alert or alarm may be made, and vehicle operation may be restricted based on the level of intoxication.

Multiple sensor assemblies (e.g., an array of sensors) may be utilized to detect which occupant among a plurality of vehicle occupants is intoxicated based on alcohol and/or carbon dioxide levels at different locations within the vehicle. When the sensor system determines that the driver is intoxicated, regardless of the intoxication of the passengers, one or more alerts may sound or be displayed, and vehicle operation may be limited. When the sensor system determines that the driver is not intoxicated while one or more passengers are intoxicated, vehicle operation may remain normal.

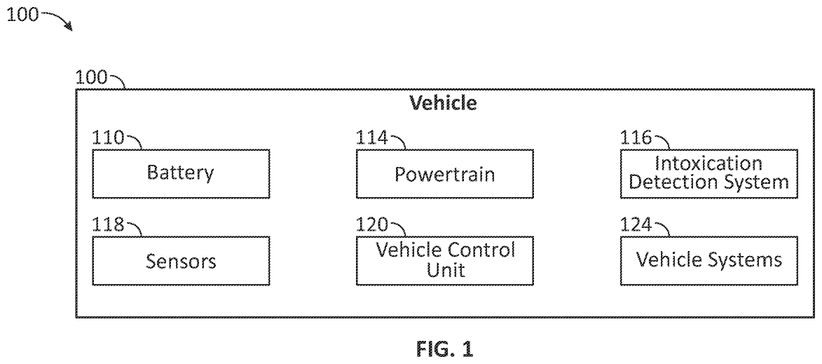

FIG. 1 is a diagram illustrating various systems of a vehicle 100, according to one or more embodiments of the disclosure. Referring to FIG. 1, vehicle 100 may include a battery 110, a powertrain 114, an intoxication detection system 116, one or more sensors 118, a vehicle control unit 120, and other vehicle systems 124, among other systems and components. Vehicle 100 may be any type of vehicle, including, for example, a truck, a sport utility vehicle, a crossover utility vehicle, a van, a multipurpose vehicle, a sedan, a hatchback, a wagon, a coupe, a sports car, a convertible, or any other type of vehicle. Vehicle 100 may be an electric vehicle or a vehicle with an internal combustion engine (e.g., a gas-powered vehicle, a diesel-powered vehicle, a hybrid vehicle, etc.), as detailed below. In embodiments, vehicle 100 is not limited to automobiles, and may include a recreational vehicle (RV), an off-highway vehicle (OHV), a side-by-side vehicle, or a golf cart, among other vehicles.

Battery 110 may be any device used as a source of electrical power, such as any device that converts chemical energy into electrical energy. For example, battery 110 may include one or more electrochemical cells with external connections for powering an electrical system of vehicle 100. Battery 110 may be rechargeable, such as by alternator 112, and may include many types or configurations, including lithium ion, lead-acid, or wet cell, among others.

Powertrain 114 is an assembly of every component operable to propel vehicle 100 into motion (e.g., moves vehicle 100 forward or in reverse). For example, powertrain 114 may harness power from a motor and/or an engine and deliver the power to one or more wheels of vehicle 100 via a drivetrain. Depending on the application, the drivetrain may include a transmission, a driveshaft, one or more axles, and a differential, among other drivetrain components, or any combination thereof.

As noted above, powertrain 114 may include an electric motor operable to power one or more wheels of vehicle 100. The motor, with may be referred to as a traction motor, may convert direct current (DC) or alternating current (AC) electrical energy, such as electrical energy provided by battery 110, into mechanical energy. For example, motor 130 may include an output shaft driven into rotation by induced magnetic fields via DC or AC current. Depending on the application, vehicle 100 may include a single motor or multiple motors.

Additionally, or alternatively, powertrain 114 may include an engine, such as an internal combustion engine, although other configurations are contemplated. For example, the engine may be a heat engine in which the combustion of a fuel occurs with an oxidizer (e.g., air) in a combustion chamber. An expansion of high-pressure gases produced by the combustion applies a force to some component of the engine (e.g., pistons, blades, rotor, nozzle, etc.), moving the component over a distance, such as to power one or more wheels of vehicle 100.

Intoxication detection system 116 may utilize various sensor inputs, algorithms, and decision structures to detect an intoxication level of one or more occupants of vehicle 100. For example, intoxication detection system 116 may include various sensors (e.g., sensor assemblies) and controllers configured to identify intoxicated (e.g., drunken) drivers and/or passengers. In embodiments, intoxication detection system 116 may monitor alcohol levels, carbon dioxide levels, or both inside the cabin of vehicle 100 to determine a blood alcohol content (BAC) of the driver and/or passenger(s). In some embodiments, intoxication detection system 116 may also monitor air quality and/or detect one or more pathogens within the vehicle cabin, such as part of a smart HVAC system or a cabin cleanliness detection system.

Intoxication detection system 116 may be configured to determine which occupant among a plurality of occupants in vehicle 100 is intoxicated, such as based on an analysis of sensor signals received from one or more (e.g., a plurality of) sensors/sensor assemblies. For example, intoxication detection system 116 may determine whether the driver of vehicle 100 is intoxicated and/or whether one or more passengers in vehicle 100 are intoxicated. When intoxication detection system 116 determines that the driver is intoxicated, intoxication detection system 116 may output an alert and/or cause a change in vehicle performance. For example, when an intoxicated driver of vehicle 100 is detected, the driver may be alerted, such as via a display of vehicle 100. On the other hand, when intoxication detection system 116 determines that only a passenger of vehicle 100 is intoxicated, no alert or vehicle change may occur.

In embodiments, alerts and/or actions performed by intoxication detection system 116 may be based on an intoxication level of the driver. For example, intoxication detection system 116 may output a first alert (e.g., a first warning symbol) based on a first intoxication level of the driver, and a second alert (e.g., a second warning symbol different from the first warning symbol) based on a second intoxication level of the driver, as described below. In embodiments, intoxication detection system 116 may provide one or more alarms, affect vehicle performance, and/or perform one or more safety functions based on the detected intoxication level of the driver. For instance, intoxication detection system 116 may lock operation of vehicle 100 and provide the driver other options, such as road assistance, ridesharing requests, etc. In some embodiments, intoxication detection system 116 may prevent shifting from park, provide one or more notifications to a mobile application and/or a network, and/or enable one or more safety features of vehicle 100. For example, intoxication detection system 116 may reduce a music volume, reenable one or more vehicle safety features turned off (e.g., lane departure warning system, collision detection and avoidance systems, etc.), reduce a maximum speed limiter, disable a vehicle ignition, or pull the vehicle off a road based on the intoxication level of the driver, as detailed more fully below.

Sensors 118 may include any number and type of sensor supporting operation of vehicle 100 or any other operations described herein (e.g., intoxication detection and/or safety mitigation). For example, sensors 118 may include an accelerometer, an inertial measurement unit (IMU), a vehicle speed sensor, an engine/motor sensor, a brake sensor, a steering sensor, or a camera, among other sensors, or any combination thereof.

Vehicle control unit 120 may be any logic device, controller, processor, module, circuitry, or device configured to perform one or more operations. Vehicle control unit 120, which may be referred to as a logic device, may be implemented as any appropriate controller (e.g., processing device, microcontroller, electronic control unit, processor, application specific integrated circuit (ASIC), field programmable gate array (FPGA), memory storage device, memory reader, or other device or combinations of devices) that may be adapted to execute, store, and/or receive appropriate instructions, such as software instructions for controlling various operations of vehicle 100, such as intoxication detection system 116, vehicle systems 124, and/or other elements of vehicle 100, for example. Such software instructions may also implement methods for processing sensor signals or data, determining sensor information, providing user feedback (e.g., through a user interface), querying devices for operational parameters, selecting operational parameters for devices, or performing any of the various operations described herein (e.g., operations performed by logic devices of various devices of vehicle 100).

Vehicle control unit 120 may be communicatively connected to intoxication detection system 116 and vehicle systems 124. Vehicle control unit 120 may be configured to receive data from at least one sensor 118 of vehicle 100. Depending on the application, vehicle control unit 120 may receive and transmit data over wired or wireless communication.

With continued reference to FIG. 1, vehicle 100 may include other components or systems. For example, vehicle systems 124 may include an onboard charger system, a power control unit, a suspension system, a GPS system, a vehicle dynamics system, a thermal system, a braking system, a steering system, and one or more safety systems. In embodiments, vehicle systems 124 may include various interfaces, controllers, and/or user interfaces.

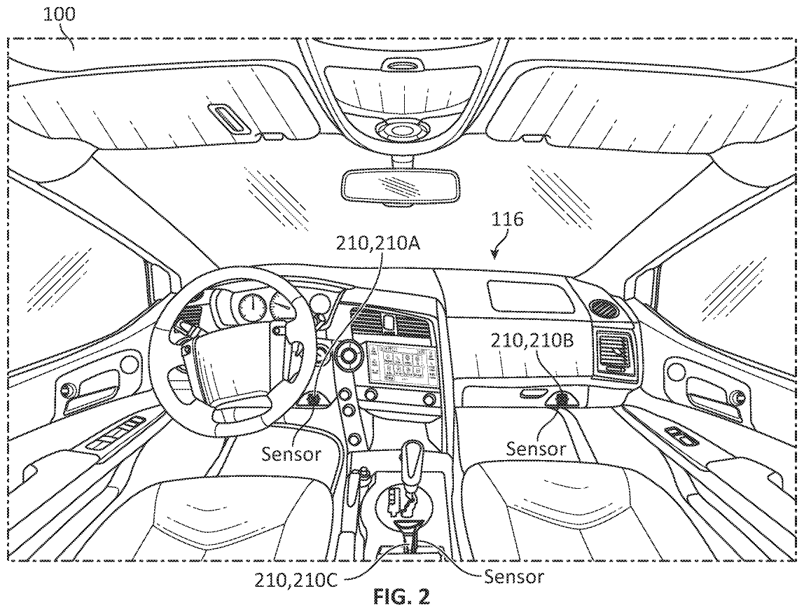

FIG. 2 is a diagram illustrating a sensor assembly placement within vehicle 100, according to one or more embodiments of the disclosure. Referring to FIG. 2, intoxication detection system 116 may include a plurality of sensor assemblies 210 disposed at different locations within vehicle 100 (e.g., within a cabin 212 of vehicle 100). For example, intoxication detection system 116 may include one or more sensor assemblies 210 placed to detect intoxication levels of the driver, and one or more sensor assemblies 210 placed to detect intoxication levels of the passenger(s). In this manner, intoxication detection system 116 may determine which occupant among multiple occupants in vehicle 100 is intoxicated based on detected levels (e.g., alcohol levels, carbon dioxide levels, etc.) at different locations.

With continued reference to FIG. 2, an array of sensor assemblies 210 may include a first sensor assembly 210A, a second sensor assembly 210B, and a third sensor assembly 210C. First sensor assembly 210A may be positioned at a first location within vehicle 100, such as in a front driver area of vehicle 100. Second sensor assembly 210B may be positioned at a second location within vehicle 100, such as in a front passenger area of vehicle 100. Third sensor assembly 210C may be positioned at a third location within vehicle 100, such as in a rear passenger area of vehicle 100. As shown, first sensor assembly 210A may be positioned at a driver kick panel of vehicle 100, second sensor assembly 210B may be positioned at a front passenger kick panel of vehicle 100, and third sensor assembly 210C may be positioned a center console of vehicle 100, such as towards or at the rear of the center console facing the rear passenger area of vehicle 100.

Such configurations are exemplary only, and sensor assemblies 210 may be positioned at other locations within vehicle 100. For example, sensor assemblies 210 may be positioned at any suitable location to detect intoxication levels of the driver and passenger(s) (e.g., front and rear passengers). Suitable locations include those within the vehicle cabin where air flow has a low disturbance regardless of window and HVAC settings. In embodiments, intoxication detection system 116 may consider whether one or more windows are open in determining the intoxication level of the driver and/or passenger(s) of vehicle 100.

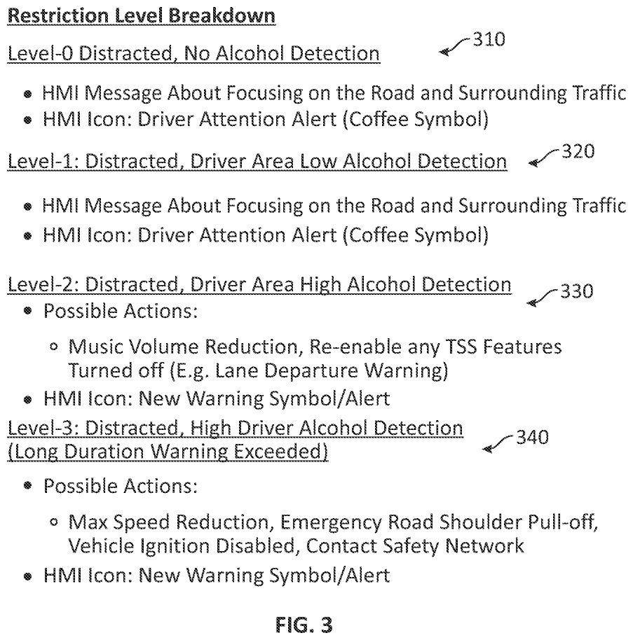

FIG. 3 is a diagram illustrating vehicle restriction levels for a vehicle, according to one or more embodiments of the disclosure. Referring to FIG. 3, intoxication detection system 116 may receive a distracted driving index determined for the driver of vehicle 100. For example, one or more systems of vehicle 100 may monitor the driver for one or more signs of distracted driving, such as vehicle wandering and/or lane departure, failing to maintain a consistent speed, sudden or frequent brake use, bent-over posture, etc. In such embodiments, intoxication detection system 116 may receive an indication that the driver is distracted and/or a level of distractedness of the driver based on the detected driving behavior.

Intoxication detection system 116 may set a vehicle restriction level for vehicle 100 based on the distracted driving index and an intoxication level of the driver (e.g., an alcohol detection for the driver). The vehicle restriction level may define one or more alerts and/or actions to be performed to reduce a current driving behavior. For example, intoxication detection system 116 may set the vehicle restriction level from a plurality of available restriction levels, with each available restriction level defining a different set of alerts and/or actions to be performed. As shown, intoxication detection system 116 may choose from a first restriction level 310 (e.g., a Level 0), a second restriction level 320 (e.g., a Level 1), a third restriction level 330 (e.g., a Level 2), or a fourth restriction level 340 (e.g., a Level 3). As poor driving continues, intoxication detection system 116 may increase the alerts and/or actions to reduce the current driving behavior. In embodiments, intoxication detection system 116 may increase the restriction level with continued poor driving behavior.

The first restriction level 310 may apply when the driver is distracted, but no alcohol is detected for the driver (e.g., in the driver area), such as when only a passenger is intoxicated. When the first restriction level 310 is set for vehicle 100, an HMI message or other indication (e.g., a display icon) may be provided to focus the driver on the road and surrounding traffic.

The second restriction level 320 may apply when the driver is distracted and a low alcohol level is detected for the driver (e.g., in the driver area). When the second restriction level 320 is set for vehicle 100, an HMI message or other indication (e.g., a display icon) may be provided to focus the driver on the road and surrounding traffic. The HMI message/icon may the same as that provided in the first restriction level 310.

The third restriction level 330 may apply when the driver is distracted and a high alcohol level is detected for the driver (e.g., in the driver area) for a short duration. When the third restriction level 330 is set for vehicle 100, a new HMI message or other indication (e.g., a display icon) may be provided, such as to alert the driver of the detected intoxication level and/or restriction level. Additionally, or alternatively, intoxication detection system 116 may reduce a music volume and/or reenable any safety features turned off (e.g., lane departure warning, etc.), among other actions, or any combination thereof.

The fourth restriction level 340 may apply when the driver is distracted and a high alcohol level is detected for the driver (e.g., in the driver area) for a long duration. When the fourth restriction level 340 is set for vehicle 100, a new HMI message or other indication (e.g., a display icon) may be provided, such as to alert the driver of the detected intoxication level and/or restriction level. Additionally, or alternatively, intoxication detection system 116 may reduce a maximum speed limiter, disable vehicle ignition, perform an emergency road shoulder pull-off maneuver, and/or contact a safety network, among other actions, or any combination thereof.

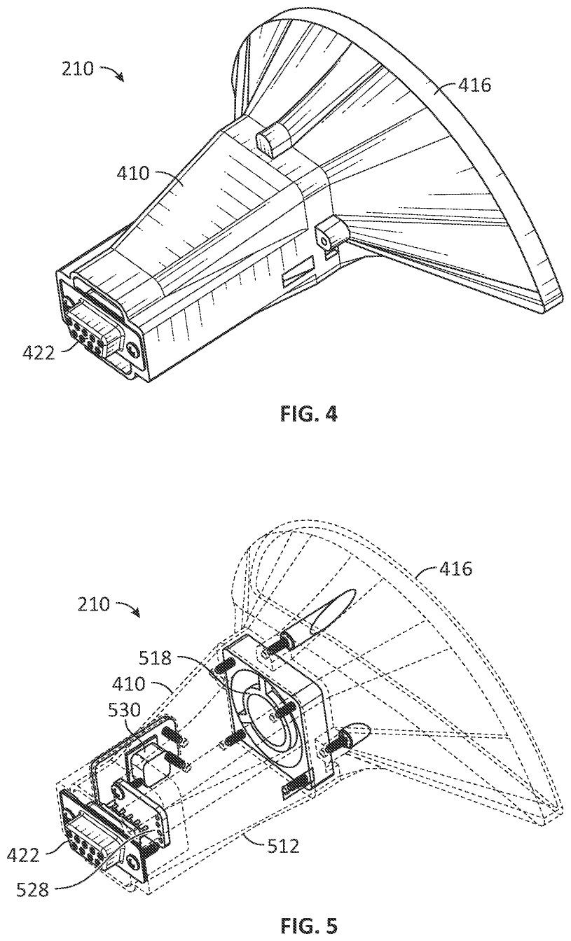

FIGS. 4-6 illustrate various views of sensor assembly 210, according to one or more embodiments of the disclosure. FIG. 4 is a diagram illustrating sensor assembly 210, according to one or more embodiments of the disclosure. Referring to FIG. 4, sensor assembly 210 includes a sensor module 410 housing one or more sensors, as detailed below. In embodiments, sensor assembly 210 may include a funneling element 416 coupled (e.g., attached) to sensor module 410, such as to an end of sensor module 410. As shown, sensor module 410 may include one or more electrical connectors 422 (e.g., a DB9 connector or any other type of connector) to electrically couple sensor module 410 to vehicle 100 (e.g., to intoxication detection system 116). As shown, electrical connector 422 may be disposed at an end of sensor module 410 opposite funneling element 416, although other configurations are contemplated.

FIG. 5 is a diagram illustrating a transparent view of sensor assembly 210, according to one or more embodiments of the disclosure. Referring to FIG. 5, sensor module 410 may define a housing 512 through which air may pass (e.g., actively or passively). In such embodiments, funneling element 416 may be configured to direct a breath sample of an occupant in vehicle 100 towards housing 512, such as towards an inlet of housing 512. As shown, funneling element 416 may be a cone housing, although other shapes and configurations are contemplated. Funneling element 416 may interface with an interior of vehicle 100. For instance, funneling element 416 may be attached to a headliner, a panel, a steering column, or a seat, among other portions of vehicle 100, in an inconspicuous manner.

In embodiments, sensor assembly 210 may include a fan 518 positioned at the inlet of housing 512 to draw the breath sample into housing 512. In embodiments, fan 518 may be positioned between housing 512 and funneling element 416. In embodiments, fan 518 may be received (e.g., recessed) within housing 512 and/or funneling element 416.

With continued reference to FIG. 5, sensor module 410 may house multiple sensors. For example, sensor assembly 210 may include an alcohol sensor 528 and a carbon dioxide sensor 530 positioned within housing 512. For example, alcohol sensor 528 may be positioned at an end of housing 512, with carbon dioxide sensor 530 positioned on an adjacent side wall of housing 512. Alcohol sensor 528 may be a fuel cell type electrochemical ethanol sensor. Carbon dioxide sensor 530 may be an NDIR carbon dioxide sensor configured to calculate a dilution factor of a breath sample. Such embodiments are illustrative only, and sensor assembly 210 may include other sensor arrangements and configurations.

Intoxication detection system 116 may analyze one or more sensor signals from alcohol sensor 528 and/or carbon dioxide sensor 530 to calculate or otherwise estimate a blood alcohol content (BAC) of one or more vehicle occupants. For instance, intoxication detection system 116 may calculate/estimate the BAC of each occupant of a plurality of occupants based on detected alcohol and carbon dioxide levels at different locations within vehicle 100. In this manner, intoxication detection system 116 may determine which occupant among a plurality of occupants in vehicle 100 is intoxicated.

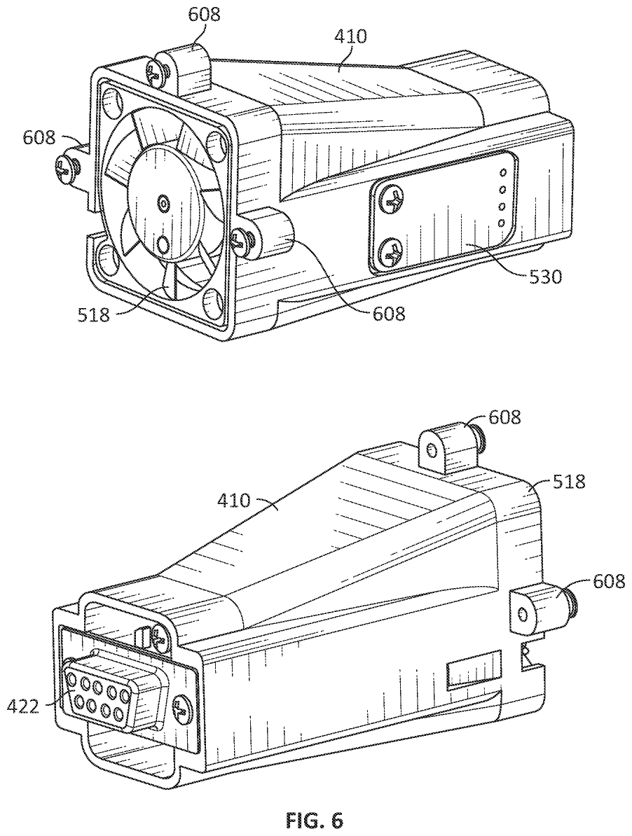

FIG. 6 is a diagram illustrating front and rear perspective views of sensor module 410, according to one or more embodiments of the disclosure. Referring to FIG. 6, fan 518 may be formed integrally (e.g., monolithically) with housing 512, with one or more mounting tabs 608 positioned at a periphery of fan 518 to connect funneling element 416 to sensor module 410. As shown, carbon dioxide sensor 530 may be inserted and secured to a side of housing 512. Alcohol sensor 528 may be inserted and secured to a terminal end 614 of housing 512, such as behind connector 422. Such embodiments are illustrative only, and sensor module 410 may include other configurations.

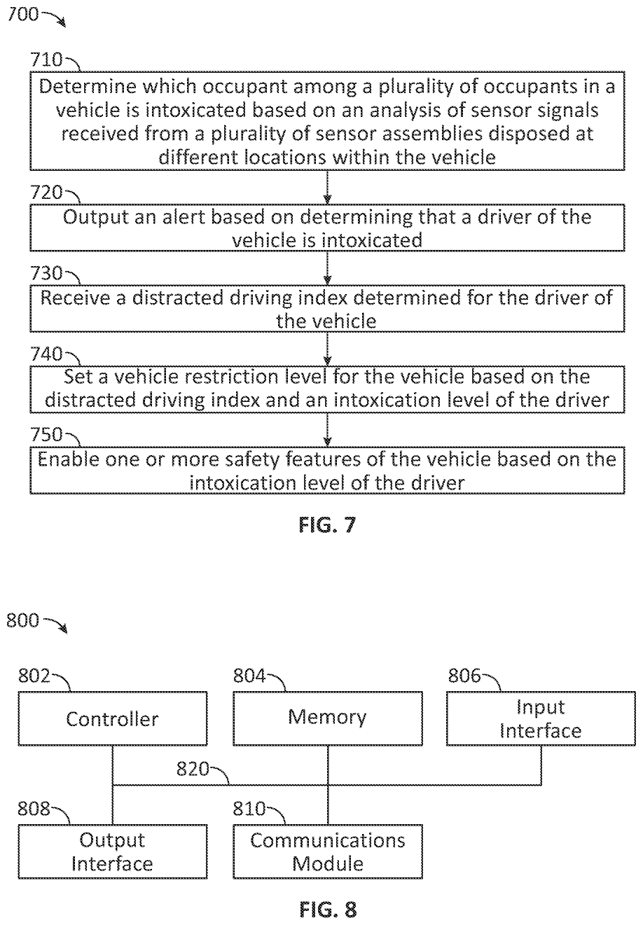

FIG. 7 is a flowchart of a method 700 of detecting an intoxication level of one or more vehicle occupants, according to one or more embodiments of the disclosure. For explanatory purposes, method 700 is described herein with reference to FIGS. 1-6, although method 700 is not limited to the embodiments illustrated in FIGS. 1-6. Note that one or more operations in FIG. 7 may be combined, omitted, and/or performed in a different order as desired.

In block 710, method 700 includes determining which occupant among a plurality of occupants in vehicle 100 is intoxicated based on an analysis of sensor signals received from a plurality of sensor assemblies disposed at different locations within vehicle 100. In embodiments, block 710 includes calculating a BAC of the plurality of occupants based on detected alcohol and carbon dioxide levels at the different locations within vehicle 100, such as in a manner as described above.

In block 720, method 700 includes outputting an alert based on determining that a driver of vehicle 100 is intoxicated. Block 720 may include outputting a first alert based on a first intoxication level of the driver, and a second alert based on a second intoxication level of the driver, such as the alerts described above.

In block 730, method 700 may include receiving a distracted driving index determined for the driver of vehicle 100. The distracted driving index may be determined based on detected signs of distracted driving, including, for instance, vehicle wandering and/or lane departure, failing to maintain a consistent speed, sudden or frequent brake use, bent-over posture, etc. The distracted driving index may be determined by intoxication detection system 116 or received by intoxication detection system 116 from one or more systems of vehicle 100 (e.g., vehicle systems 124).

In block 740, method 700 may include setting a vehicle restriction level for vehicle 100 based on the distracted driving index and an intoxication level of the driver. For example, block 740 may include selecting a restriction level from among a plurality of restriction levels based on the distracted driving index and the intoxication level of the driver. The vehicle restriction levels may be similar to those described above with reference to FIG. 3.

In block 750, method 700 may include enabling one or more safety features of vehicle 100 based on the intoxication level of the driver. For instance, block 750 may include reducing a music volume, reenabling one or more vehicle safety features, reducing a maximum speed limiter, disabling a vehicle ignition, or pulling vehicle 100 off a road based on the intoxication level of the driver, among other actions, such as in a manner as detailed above.

FIG. 8 is a diagram illustrating an example computing or processing system 800 in which embodiments of the present disclosure may be implemented, according to one or more embodiments of the disclosure. For example, intoxication detection system 116, described above, may be implemented using system 800. In some embodiments, method 700 of FIG. 7, described above, may be implemented using system 800. System 800 can be or include a computer, phone, PDA, tablet, server, controller, or any other type of electronic device. Such an electronic device includes various types of computer readable media and interfaces for various other types of computer readable media. As shown in FIG. 8, system 800 includes a controller 802, a memory 804, an input interface 806, an output interface 808, and a communications module 810.

Controller 802, according to various embodiments, includes one or more of a processor, a microprocessor, a central processing unit (CPU), an electronic control unit, a graphics processing unit (GPU), a single-core processor, a multi-core processor, a microcontroller, a programmable logic device (PLD) (e.g., field programmable gate array (FPGA)), an application specific integrated circuit (ASIC), a digital signal processing (DSP) device, or other logic device that may be configured, by hardwiring, executing software instructions, or a combination of both, to perform various operations discussed herein for embodiments of the disclosure. Controller 802 may be configured to interface and communicate with the various other components of system 800 to perform such operations. For example, controller 802 may be configured to receive and process data received from a network and/or one or more sensors (e.g., sensors 118), store the data in memory 804, and/or retrieve stored data from memory 804.

Controller 802 may include combinations of hardware and software processing functionality and may be provided with/in and/or communicatively attached to other components to execute appropriate instructions, such as software instructions and/or processing parameters stored in memory 804. In various embodiments, controller 802 may be configured to execute software instructions stored in memory 804 to perform various methods, processes, or operations in the manner described herein.

Memory 804 includes, in one embodiment, one or more memory devices configured to store data and information, including magnetic flux data and position information. The memory 804 may include one or more various types of memory devices including volatile and non-volatile memory devices, such as random-access memory (RAM), dynamic RAM (DRAM), static RAM (SRAM), non-volatile random-access memory (NVRAM), read-only memory (ROM), programmable read-only memory (PROM), erasable programmable read-only memory (EPROM), electrically-erasable programmable read-only memory (EEPROM), flash memory, hard disk drive, and/or other types of memory. As discussed above, controller 802 may be configured to execute software instructions stored in memory 804 to perform method 700 and process steps and/or operations. Controller 802 may be configured to store data in memory 804.

Input interface 806 includes, in one embodiment, a user input and/or an interface device, such as one or more controls, knobs, buttons, slide bars, keyboards, sensors, cameras, and/or other devices, that are adapted to generate an input control signal. Controller 802 may be configured to sense the input control signals from input interface 806 and respond to any sensed input control signals received therefrom. Controller 802 may be configured to interpret such an input control signal as a value, as generally understood by one skilled in the art. In one embodiment, input interface 806 may include a control unit (e.g., a wired or wireless handheld control unit) having push buttons adapted to interface with a user and receive user input control values. In one implementation, the push buttons of the control unit may be used to control various system functions.

Output interface 808 may enable, for example, the output of data or other information. Output interface 808 may include, for example, one or more display devices, such as monitors or other visual displays (e.g., light emitting diode (LED) displays, liquid crystal displays (LCDs), head-up displays (HUDs), or other types of displays). Some implementations include devices such as a touchscreen that function as both input and output components. Controller 802 may be configured to render data and information on output interface 808. For example, controller 802 may be configured to render data on output interface 808, such as data stored in memory 804.

In some embodiments, various components of system 800 may be distributed and in communication with one another over a network. In this regard, communications module 810 may be configured to facilitate wired and/or wireless communication among various system components over the network. Such a network may include, for example, a local area network (“LAN”), such as an Intranet, or a wide area network (“WAN”), such as the Internet.

In embodiments, various components of system 800 may be communicatively connected via a system communications bus 820. Bus 820 collectively represents all system, peripheral, and chipset buses that communicatively connect the numerous devices of system 800. For instance, bus 820 may communicatively connect controller 802, memory 804, input interface 806, output interface 808, communications module 810, powertrain torque control system 116, or any combination thereof, together.

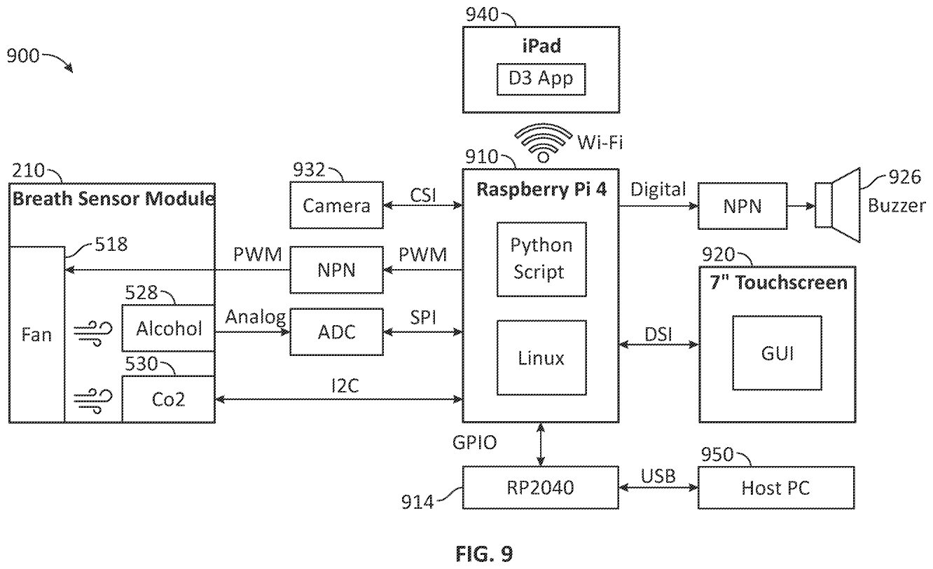

FIG. 9 is a diagram illustrating an example system 900 in which embodiments of the present disclosure may be implemented, according to one or more embodiments of the disclosure. For example, intoxication detection system 116, system 800, and/or method 700, described above, may be implemented using system 900. Referring to FIG. 9, system 900 includes sensor assembly 210, a processing board 910, and a controller 914. In embodiments, system 900 may include a display 920, a buzzer 926, and a camera 932, or any combination thereof.

Processing board 910 may include combinations of hardware and software processing functionality and may be provided with/in and/or communicatively attached to other components to execute appropriate instructions, such as software instructions and/or processing parameters stored in memory. In various embodiments, processing board 910 may be configured to execute software instructions stored in memory to perform various methods, processes, or operations in the manner described herein.

Processing board 910 may be coupled to sensor assembly 210 to control operation of fan 518 and/or receive signals from alcohol sensor 528 and carbon dioxide sensor 530 (e.g., via connector 422). For example, processing board 910 may be coupled to fan 518 via a PWM connection, such as through a negative-positive-negative (NPN) transistor, although other configurations are contemplated. As shown, processing board 910 may be coupled to alcohol sensor 528 via a serial peripheral interface (SPI) and analog-to-digital converter (ADC), although other configurations are contemplated. Processing board 910 may be coupled to carbon dioxide sensor 530 using an I2C communication protocol, although other configurations are contemplated.

Controller 914 and/or processing board 910 may include one or more of a processor, a microprocessor, a central processing unit (CPU), an electronic control unit, a graphics processing unit (GPU), a single-core processor, a multi-core processor, a microcontroller, a programmable logic device (PLD) (e.g., field programmable gate array (FPGA)), an application specific integrated circuit (ASIC), a digital signal processing (DSP) device, or other logic device that may be configured, by hardwiring, executing software instructions, or a combination of both, to perform various operations discussed herein for embodiments of the disclosure. Controller 914 may be configured to interface and communicate with the various other components of system 900 to perform such operations. For example, controller 914 may be configured to receive and process data received from a network and/or one or more sensors (e.g., sensor assembly 210), store the data in memory, and/or retrieve stored data from memory. In embodiments, controller 914 may include a communications module configured to facilitate wired and/or wireless communication among various system components over the network, such as an external device 940 (e.g., via a local area network (“LAN”), such as an Intranet, or a wide area network (“WAN”), such as the Internet).

As shown, controller 914 may be coupled to processing board 910 via a general-purpose input/output (GPIO), such as an uncommitted digital signal pin on an integrated circuit or electronic circuit board controllable by controller 914, although other configurations are contemplated. In embodiments, controller 914 may be coupled to a host PC 950, such as via a USB connection.

Display 920 may be coupled to processing board 910 (e.g., via a digital serial interface) and may include one or more display elements or user interfaces. For example, display 920 may be a touchscreen including a graphical user interface (GUI) allowing touch control operation of system 900 (or various components of system 900) via display 920.

Buzzer 926 may be coupled to processing board 910 (e.g., via an NPN transistor) and provide an audible alarm. For instance, detection of an intoxicated driver in vehicle 100 may trigger buzzer 926 to sound an alarm, warning the driver and other occupants of vehicle 100 of the detected intoxication. The alarms provided by buzzer 926 may vary based on the detected intoxication level of the driver and/or passenger(s).

Camera 932 may be coupled to processing board 910 and provide one or more images of the occupants of vehicle 100. For example, one or more images of the driver may be captured by camera 932 to facilitate a determination of a distracted driving index for the driver, as described above. In embodiments, images captured by camera 932 may be used to determine or support a determination that the driver or passenger(s) are intoxicated.

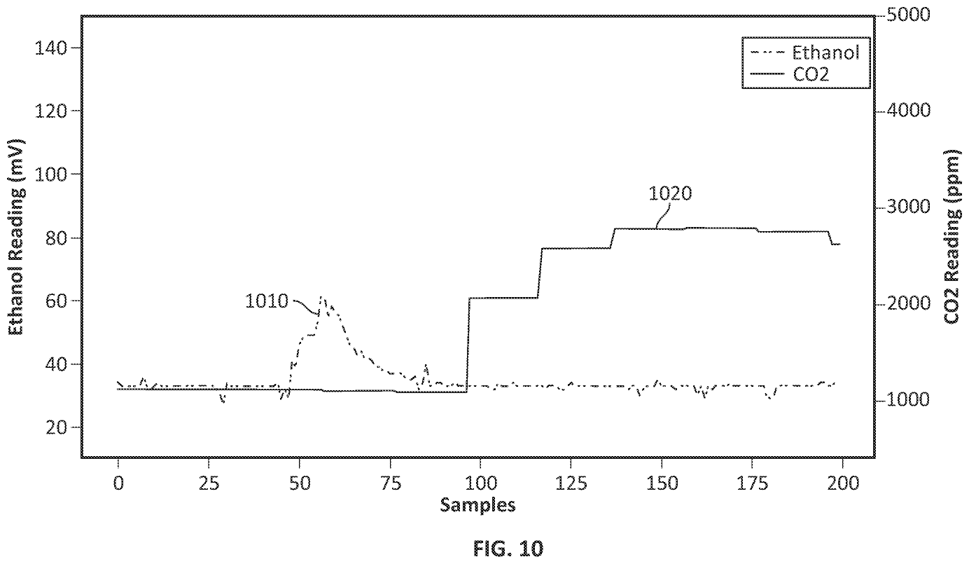

FIG. 10 is a diagram illustrating example sensor readings of sensor assembly 210, according to one or more embodiments of the disclosure. Referring to FIG. 10, a controller or logic device (e.g., intoxication detection system 116, controller 802, processing board 910 and/or controller 914, etc.) may receive one or more sensor signals from sensor assembly 210, such as an alcohol reading 1010 and/or a carbon dioxide reading 1020. Alcohol reading 1010 may be taken at a first sample rate, and carbon dioxide reading 1020 may be taken at a second sample rate. Depending on the application, the first and second sample rates may be similar or different. When similar, a spike in alcohol and carbon dioxide detection may line up in the graph. When samples are taken at different rates, spikes in alcohol and carbon dioxide detection may be offset, such as that shown in FIG. 10. In any case, an increased alcohol (e.g., ethanol) reading and/or an increased carbon dioxide reading may indicate that an occupant is intoxicated based on the detected values.

Where applicable, various embodiments provided by the present disclosure can be implemented using hardware, software, or combinations of hardware and software. Also, where applicable, the various hardware components and/or software components set forth herein can be combined into composite components comprising software, hardware, and/or both without departing from the spirit of the present disclosure. Where applicable, the various hardware components and/or software components set forth herein can be separated into sub-components comprising software, hardware, or both without departing from the spirit of the present disclosure. In addition, where applicable, it is contemplated that software components can be implemented as hardware components, and vice-versa.

Software in accordance with the present disclosure, such as non-transitory instructions, program code, and/or data, can be stored on one or more non-transitory machine-readable mediums. It is also contemplated that software identified herein can be implemented using one or more general purpose or specific purpose computers and/or computer systems, networked and/or otherwise. Where applicable, the ordering of various steps described herein can be changed, combined into composite steps, and/or separated into sub-steps to provide features described herein.

While certain exemplary embodiments of the invention have been described and shown in the accompanying drawings, it is to be understood that such embodiments are merely illustrative of and not restrictive on the broad invention, and that the embodiments of the invention not be limited to the specific constructions and arrangements shown and described, since various other modifications may occur to those ordinarily skilled in the art. The intention is to cover all modifications, equivalents and alternatives falling within the spirit and scope of the disclosure as defined by the claims.

For example, the elements and teachings of the various embodiments may be combined in whole or in part in some or all of the embodiments. In addition, one or more of the elements and teachings of the various embodiments may be omitted, at least in part, and/or combined, at least in part, with one or more of the other elements and teachings of the various embodiments. In addition, while different steps, processes, and procedures are described as appearing as distinct acts, one or more of the steps, one or more of the processes, and/or one or more of the procedures may also be performed in different orders, simultaneously, and/or sequentially. In some embodiments, the steps, processes, and/or procedures may be merged into one or more steps, processes, and/or procedures. In some embodiments, one or more of the operational steps in each embodiment may be omitted.

1. A system comprising:
a plurality of sensor assemblies disposed at different locations within a vehicle; and
a controller configured to:
determine which occupant among a plurality of occupants in the vehicle is intoxicated based on an analysis of sensor signals received from the plurality of sensor assemblies, and
output an alert based on determining that a driver of the vehicle is intoxicated, wherein the controller is configured to reduce a music volume or pull the vehicle off a road based on the intoxication level of the driver.

2. The system of claim 1, wherein the controller is configured to:
output a first alert based on a first intoxication level of the driver;
output a second alert based on a second intoxication level of the driver; and
output a third alert based on a third intoxication level of the driver,
wherein the first, second and third alerts are different from one another.

3. The system of claim 1, wherein the controller is configured to enable one or more safety features of the vehicle based on an intoxication level of the driver.

4. (canceled)

5. The system of claim 1, wherein the controller is configured to:
receive a distracted driving index determined for the driver of the vehicle; and
set a vehicle restriction level for the vehicle based on the distracted driving index and an intoxication level of the driver, wherein the vehicle restriction level defines one or more alerts and/or actions to be performed to reduce a current driving behavior.

6. The system of claim 1, wherein:
each sensor assembly of the plurality of sensor assemblies comprises an alcohol sensor and a carbon dioxide sensor; and
the controller is configured to calculate a blood alcohol content of the plurality of occupants based on detected alcohol and carbon dioxide levels at the different locations.

7. The system of claim 1, wherein at least one sensor assembly of the plurality of sensor assemblies comprises:
a housing;
a funneling element configured to direct a breath sample of an occupant in the vehicle towards the housing; and
a fan operable to draw the breath sample into the housing.

8. A vehicle comprising:
a cabin;
a plurality of sensor assemblies disposed at different locations within the cabin; and
a controller configured to:
determine which occupant among a plurality of occupants in the cabin is intoxicated based on an analysis of sensor signals received from the plurality of sensor assemblies, and
output an alert based on determining that a driver of the vehicle is intoxicated, wherein the controller is configured to reduce a music volume or pull the vehicle off a road based on the intoxication level of the driver.

9. The vehicle of claim 8, wherein the controller is configured to:
output a first alert based on a first intoxication level of the driver;
output a second alert based on a second intoxication level of the driver; and
output a third alert based on a third intoxication level of the driver,
wherein the first, second and third alerts are different from one another.

10. The vehicle of claim 8, wherein the controller is configured to enable one or more safety features of the vehicle based on an intoxication level of the driver.

11. (canceled)

12. The vehicle of claim 8, wherein the controller is configured to:
receive a distracted driving index determined for the driver of the vehicle; and
set a vehicle restriction level for the vehicle based on the distracted driving index and an intoxication level of the driver, wherein the vehicle restriction level defines one or more alerts and/or actions to be performed to reduce a current driving behavior.

13. The vehicle of claim 8, wherein:
each sensor assembly of the plurality of sensor assemblies comprises an alcohol sensor and a carbon dioxide sensor; and
the controller is configured to calculate a blood alcohol content of the plurality of occupants based on detected alcohol and carbon dioxide levels at the different locations.

14. The vehicle of claim 8, wherein at least one sensor assembly of the plurality of sensor assemblies comprises:
a housing;
a funneling element configured to direct a breath sample of an occupant in the vehicle towards the housing, the funneling element interfacing with a portion of the cabin; and
a fan operable to draw the breath sample into the housing.

15. A controller configured to:
determine which occupant among a plurality of occupants in a vehicle is intoxicated based on an analysis of sensor signals received from a plurality of sensor assemblies disposed at different locations within the vehicle; and
output an alert based on determining that a driver of the vehicle is intoxicated,
wherein the controller is configured to:
enable one or more safety features of the vehicle based on an intoxication level of the driver, and
reduce a music volume or pull the vehicle off a road based on the intoxication level of the driver.

16. The controller of claim 15, wherein the controller is configured to:
output a first alert based on a first intoxication level of the driver;
output a second alert based on a second intoxication level of the driver; and
output a third alert based on a third intoxication level of the driver,
wherein the first, second and third alerts are different from one another.

17. (canceled)

18. (canceled)

19. The controller of claim 15, wherein the controller is configured to:
receive a distracted driving index determined for the driver of the vehicle; and
set a vehicle restriction level for the vehicle based on the distracted driving index and an intoxication level of the driver, wherein the vehicle restriction level defines one or more alerts and/or actions to be performed to reduce a current driving behavior.

20. The controller of claim 15, wherein the controller is configured to calculate a blood alcohol content of the plurality of occupants based on detected alcohol and carbon dioxide levels at the different locations.
